(19) United States
(12) Patent Application Publication
(10) Pub. No.: US 2025/0057424 A1
(43) Pub. Date: Feb. 20, 2025

(54) SYSTEMS AND METHODS FOR FUNCTIONAL TASK PREDICTION USING DYNAMIC SUPERVOXEL PARCELLATIONS

(71) Applicant: University of Florida Research Foundation, Inc., Gainesville, FL (US)

(72) Inventors: Ruogu Fang, Gainesville, FL (US); Kyle B. See, Gainesville, FL (US); Stephen Coombes, Gainesville, FL (US)

(21) Appl. No.: 18/720,869

(22) PCT Filed: Dec. 16, 2022
(86) PCT No.: PCT/US2022/081801

§ 371 Date: Jun. 17, 2024

Related U.S. Application Data
(60) Provisional application No. 63/290,224, filed on Dec. 16, 2021.

(51) Int. Cl. A61B 5/00; A61B 5/055

(52) U.S. Cl. CPC A61B 5/0042; A61B 5/055; A61B 5/7267; A61B 2576/026

(57) ABSTRACT

Various examples are provided related to dynamic brain parcellation. In one example, a method for functional task prediction with dynamic supervoxel parcellation includes preprocessing activation data obtained from brains of multiple subjects to generate one or more dynamic parcellated supervoxel maps of the brain, the activation data associated with a functional task, and determining an anatomical location of the functional task in the brain of another subject based upon classification of supervoxels of the one or more dynamic parcellated supervoxel maps. In another example, a system includes at least one computing device that can preprocess activation data to generate one or more dynamic parcellated supervoxel maps of the brain, the activation data associated with a functional task, and determine an anatomical location of the functional task based upon classification of supervoxels of the one or more dynamic parcellated supervoxel maps.

### CROSS REFERENCE TO RELATED APPLICATIONS

This application claims priority to, and the benefit of, co-pending U.S. provisional application entitled “Systems and Methods for Functional Task Prediction Using Dynamic Supervoxel Parcellations” having Ser. No. 63/290,224, filed Dec. 16, 2021, which is hereby incorporated by reference in its entirety.

### STATEMENT REGARDING FEDERALLY SPONSORED RESEARCH OR DEVELOPMENT

This invention was made with government support under Grant Numbers UL1TR001427 and TL1TR001428, awarded by the National Institutes of Health; and Grant No. 1908299, awarded by the National Science Foundation. The government has certain rights in the invention.

### BACKGROUND

Somatotopy is the point-to-point mapping of an area of the body to a specific point in the central nervous system. Electrical stimulation on the surface of the cortex has been used to map somatotopic organization in the human brain. Within both the primary motor cortex (M1) and primary somatosensory cortex (S1), lower extremities are located dorsally and close to the midline, the arms and hands are representation more laterally, and the face representation is most lateral and ventral. Somatotopic organization is not limited to the pre- and post-central gyri (M1/S1), with neuroimaging methods such as fMRI and EEG studies revealing somatotopy in the supplementary motor area (SMA), cingulate motor area, premotor cortex, superior and inferior parietal lobules, insula, basal ganglia, and cerebellum. This raises several experimental questions: To what extent is activation in the sensorimotor network effector-dependent and effector-independent activation? How important is the sensorimotor cortex when predicting the motor effector? Is there redundancy in the distributed somatotopically organized network such that removing one region has little impact on classification accuracy? Current knowledge is unfortunately derived from experimental paradigms which often do not measure or control motor performance across effectors. This can be problematic given that the blood-oxygen-level-dependent (BOLD) signal can be influenced by differing characteristics of force production, and different effectors inherently produce different amounts of force even when flexing/extending at the same frequency.

### SUMMARY

Aspects of the present disclosure are related to dynamic brain parcellation. In one aspect, among others, a method for functional task prediction with dynamic supervoxel parcellation comprises preprocessing activation data obtained from brains of multiple subjects to generate one or more dynamic parcellated supervoxel maps based upon a whole brain map of the multiple subjects, the activation data associated with a functional task; and determining an anatomical location of the functional task in a brain of another subject based upon classification of supervoxels of the one or more dynamic parcellated supervoxel maps. In one or more aspects, the preprocessing can comprise registering and averaging the activation data of the multiple subjects to produce the whole brain map; and generating the one or more dynamic parcellated supervoxel maps from the whole brain map. Supervoxels of the one or more dynamic parcellated supervoxel maps can be identified from the whole brain map. The supervoxels can be identified using different statistical and machine learning methods such as, but not limited to, a 1-way ANOVA analysis, decision trees, artificial neural networks, and/or support vector machines. The dynamic supervoxel parcellation generated is transferrable from one subject to another making it generalizable to new population for somatotopy studies.

In various aspects, the method can comprise masking the one or more dynamic parcellated supervoxel maps using a conjunction map. The one or more dynamic parcellated supervoxel maps can comprise average beta coefficients. The one or more dynamic parcellated supervoxel maps can comprise average beta coefficients. The functional task can be associated with a foot, a hand, or a mouth of the subject or other functional tasks that can be observed. The activation data can be acquired through neuroimaging approaches such as, but not limited to, magnetic resonance imaging of the subject. In some aspects, the classification of the supervoxels can comprise generating weights for the supervoxels using different machine learning methods. The machine learning can comprise, but is not limited to, gradient boosting decision trees, artificial neural networks, or support vector machines. The anatomical location of the functional task can be determined based upon the generated weights.

In another aspect, a system comprises at least one computing device comprising processing circuitry including a processor and memory, the at least one computing device configured to at least: preprocess activation data obtained from brains of multiple subjects to generate one or more dynamic parcellated supervoxel maps based on a whole brain of the multiple subjects, the activation data associated with a functional task; and determine an anatomical location of the functional task in the brain of another subject based upon classification of supervoxels of the one or more dynamic parcellated supervoxel maps. In one or more aspects, preprocessing the activation data can comprise registering and averaging the activation data of the multiple subjects to produce the whole brain map; and generating the one or more dynamic parcellated supervoxel maps from the whole brain map. Preprocessing can comprise masking the one or more dynamic parcellated supervoxel maps using a conjunction map.

In various aspects, the one or more dynamic parcellated supervoxel maps can comprise average beta coefficients. The activation data can be acquired through magnetic resonance imaging or other neuroimaging approach of the subject while carrying out the functional task. The functional task can be associated with, but is not limited to, a foot, a hand, or a mouth of the subject. In some aspects, the classification of the supervoxels can comprise generating weights for the supervoxels using different machine learning methods. The machine learning can comprise, but is not limited to, gradient boosting decision trees, artificial neural networks, or support vector machines. The anatomical location of the functional task can be determined based upon the generated weights.

Other systems, methods, features, and advantages of the present disclosure will be or become apparent to one with skill in the art upon examination of the following drawings and detailed description. It is intended that all such additional systems, methods, features, and advantages be included within this description, be within the scope of the present disclosure, and be protected by the accompanying claims. In addition, all optional and preferred features and modifications of the described embodiments are usable in all aspects of the disclosure taught herein. Furthermore, the individual features of the dependent claims, as well as all optional and preferred features and modifications of the described embodiments are combinable and interchangeable with one another.

### BRIEF DESCRIPTION OF THE DRAWINGS

Many aspects of the present disclosure can be better understood with reference to the following drawings. The components in the drawings are not necessarily to scale, emphasis instead being placed upon clearly illustrating the principles of the present disclosure. Moreover, in the drawings, like reference numerals designate corresponding parts throughout the several views.

FIGS. 1A-2C illustrate examples of (A) a custom-designed foot force device (left) with force generated by dorsiflexion of the right foot and measured by the force transducer; (B) a hand force device which measured right hand pinch force; and (C) a custom-designed bite device with force generated against the bite plates held in the subject's mouth, and measured by the force transducer, in accordance with various embodiments of the present disclosure.

FIG. 1D illustrates an example of the task paradigm comprising a pattern of 4 s force and 2 s rest, repeated 5 times during a 30 s force block, in accordance with various embodiments of the present disclosure.

FIG. 2 illustrates an example of a process pipeline for preprocessing and classification for dynamic brain parcellation, in accordance with various embodiments of the present disclosure.

FIG. 3 illustrates an example of 5-fold nested cross validation, in accordance with various embodiments of the present disclosure.

FIG. 4 is a schematic block diagram of an example of a computing device that can be utilized for dynamic brain parcellation, in accordance with various embodiments of the present disclosure.

FIGS. 5A-5D illustrate examples of whole brain and cerebellum activation maps and 1-way ANOVAs, in accordance with various embodiments of the present disclosure.

FIG. 6 illustrates examples of whole brain and cerebellum overlap maps, in accordance with various embodiments of the present disclosure.

FIGS. 7A and 7B illustrate examples of accuracy scores across supervoxels and F1 scores across labels, in accordance with various embodiments of the present disclosure.

FIG. 8 Illustrates examples of confusion matrices at different supervoxel models, in accordance with various embodiments of the present disclosure.

FIGS. 9A and 9B illustrate examples of regions identified by classification analysis, in accordance with various embodiments of the present disclosure.

### DETAILED DESCRIPTION

Disclosed herein are various examples related to dynamic brain parcellation. A dynamic data-driven supervoxel-based brain parcellation approach offers a new and flexible framework for analyzing motor movement and various other neurological diseases and functions. The supervoxels can be identified using different statistical and machine learning methods such as, but not limited to, a 1-way ANOVA analysis, decision trees, artificial neural networks, and/or support vector machines. The dynamic supervoxel parcellation generated is transferrable from one subject to another making it generalizable to new population for somatotopy studies. Reference will now be made in detail to the description of the embodiments as illustrated in the drawings, wherein like reference numbers indicate like parts throughout the several views.

Previous studies have used brain signals to classify different effectors with varying levels of success. Although studies vary in which effectors are assessed, what movements are made, and how the movements are measured, classification performance is higher at the individual level as compared to the group level. Classifying effectors at the individual level is helpful in the context of brain-computer interfaces when there is an adaptation phase between the machine and the brain, but this approach does not always generalize well to new individuals. Other fMRI studies have implemented multivoxel pattern analyses (MVPA) to classify movements based on predefined regions of interest. While effective in determining how well the BOLD signal can differentiate effectors within a specific brain region, it is challenging to implement this approach across the whole brain using a data-driven approach.

To address these limitations, fMRI was used to assess BOLD signal changes across the whole brain while force production was precisely controlled across three different effectors: the foot, hand, and mouth. Other neuroimaging methods such as, e.g., EEG can also be utilized. Real-time visual feedback was provided to subjects while they completed a target matching task by executing a sustained isometric contraction with each effector. Conventional univariate analyses were first used to identify effector-dependent and effector-independent brain activation during task performance. Next, a novel machine learning approach was implemented which allowed control of the brain parcellation resolution by tuning the maximum number of features fed into the classifier, in order to understand how data-driven, adaptive brain parcellation granularity altered classification accuracy and key region identification. The findings provide new insight into the functional neuroanatomy required to predict the motor effector used in a motor control task. The utility of dynamic supervoxel parcellation systems and methods are not limited to this application, but can also be extended to other functional tasks that can be observed.

### Methodology

Subjects. This study included 20 right-handed adults (age=20.50±0.92 years; female=10). All participants had normal or corrected to normal vision, self-reported no disability or neurological conditions that would prevent them from completing the required force tasks with their foot, hand, or mouth. All participants provided written informed consent before testing, and all procedures were approved by the Institutional Review Board at the University of Florida.

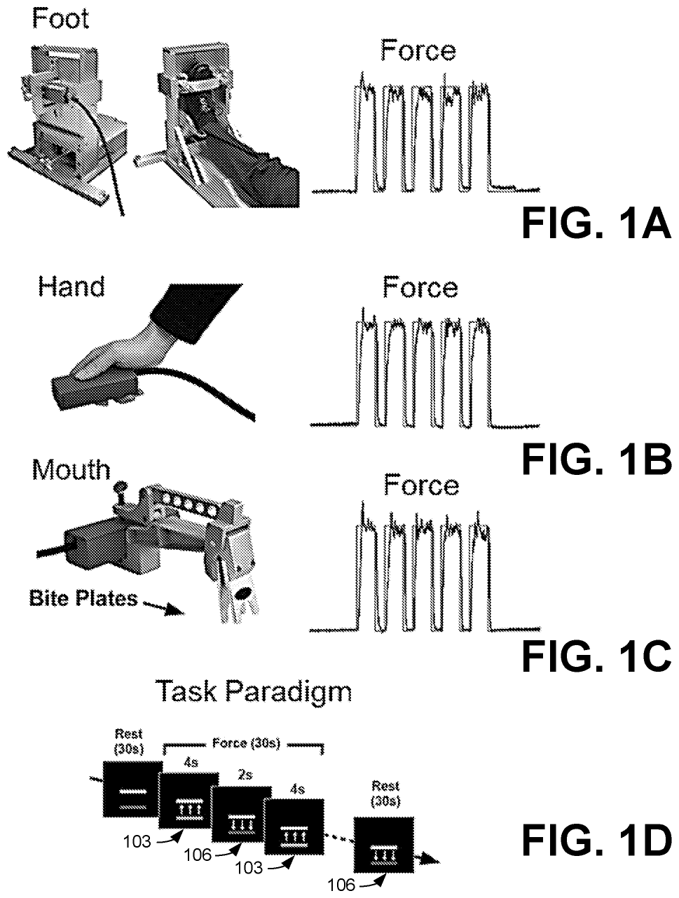

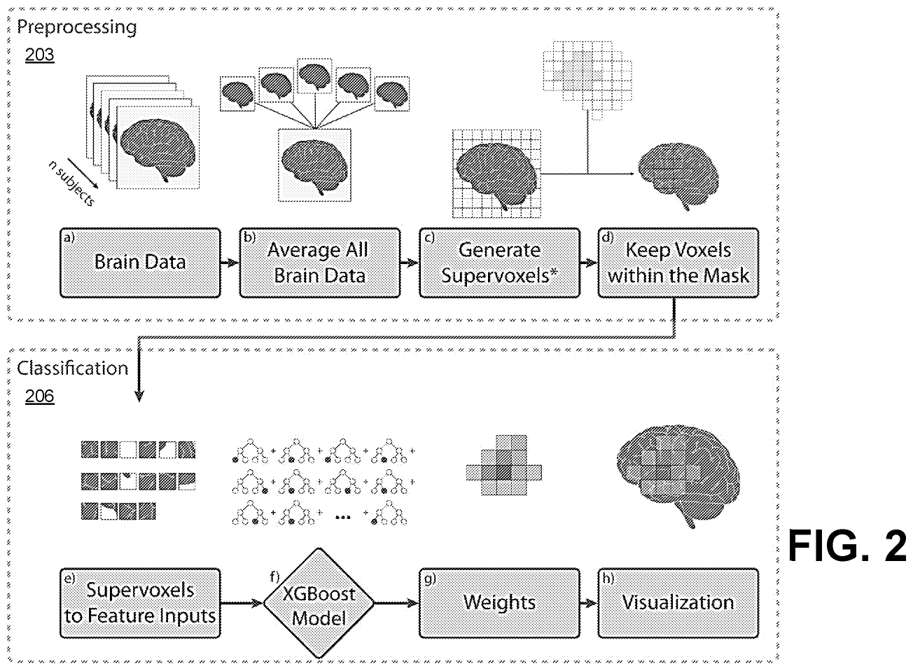

Force task and acquisition. Three force production tasks were performed by each participant inside an MRI scanner. Participants were supine for all conditions, and generated force against a custom fiber-optic force transducer with a resolution of 0.025 N (Neuroimaging Solutions, Gainesville, FL) using the right foot (see FIG. 1A), the right hand (FIG. 1B), and bilaterally by the mouth (FIG. 1C). In each, an exemplary force time series (right) shows the 10% MVC target force, as well as the force produced. Force signals were transmitted through a fiber-optic cable to an SM130 Optical Sensing Interrogator (Micron Optics, Atlanta, Georgia). The interrogator digitized analog force data at 62.5 Hz. A customized program written in LabVIEW (National Instruments, Austin, TX) collected the force data. Online visual feedback of the force output was visible to the participants at a 60 Hz refresh rate. Force data were low-pass filtered using a Butterworth, 20 Hz 4^{th}-order dual-pass filter.

Prior to entering the scanner, each participant's maximum voluntary contraction (MVC) was recorded for each effector (foot, hand, mouth) during a practice session. During a series of three consecutive MVC trials, participants held a maximum force contraction (MFC) for 5 seconds. A 60 second rest period was provided between trials. Average force during each sustained MFC was calculated and the mean of three trials was used as the participant's MVC value. fMRI data were collected while each participant produced force to a target level set at 10% of their MVC for each of the three effectors separately. Effector order was counterbalanced across participants.

A representation of the task paradigm is shown in FIG. 1D. Each task involved both a rest and force condition. During the force condition, two horizontal bars were displayed which represented the cue to produce force (green bar) 103 as well as the target force (white bar). Subjects produced force for 4 s when the bar turned green 103. The force produced caused the green bar to move vertically, providing visual feedback to the participant. The white target bar remained stationary and represented the participant's 10% MVC target. When the bar turned red 106, subjects rested for 2 s. This pattern of 4 s force and 2 s rest repeated 5 times over the 30 s force block. In the rest block, subjects rested the entire 30 s with stationary bars on the screen. Each scan started and ended with a rest block and contained a total of 4 force blocks.

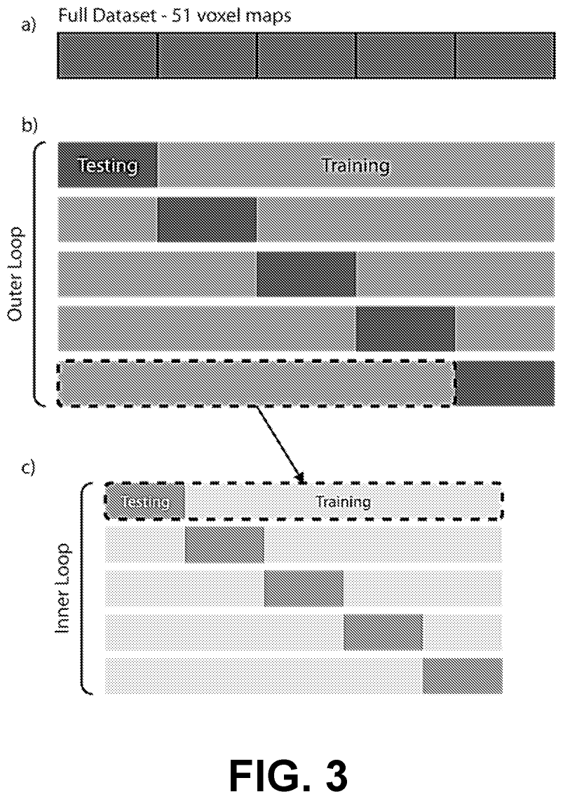

FIG. 2 illustrates a process pipeline to generate a uniform supervoxel map which is used for all subjects. The pipeline includes preprocessing 203 and classification 206. In the preprocessing 203 of FIG. 2, all subject data 203(a) are registered and averaged to produce a single whole brain map 203(b). This can ensure consistency between subjects. Supervoxel maps 203(c) can then be generated using the single whole brain map. This step onwards can be repeated for any resolution of supervoxels from the same single whole brain map. Supervoxels 203(d) are masked using a conjunction map to limit analysis on brain regions. This will affect generated supervoxels by removing all or a portion of voxels.

MRI acquisition. MRI images were collected using a 32-channel head coil inside a Phillips 3T magnetic resonance scanner (Achieva, Best, the Netherlands). T1—weighted images (resolution: 1 mm isotropic, repetition time: TR=6.8 ms, echo time: TE=3.3 ms, flip angle=8°, field of view=240 mm³, and acquisition matrix=240×240) were collected in 170 contiguous axial slices. Functional data were collected in 46 contiguous axial slices using a single-shot gradient echo-planar imaging pulse sequence (resolution: 3 mm isotropic, TR=2500 ms, TE=30 ms, flip angle=80°, field of view=240 mm³, and acquisition matrix=80×80).

Force Data Analysis. Force data were analyzed using customized algorithms in LabVIEW. Mean force generated as a percentage of MVC, variability of force generated, and target force error were calculated for each trial and then averaged separately for each effector (hand, foot, mouth). These measures were calculated from data extracted from the middle 2 s of each 4 s force pulse. Variability of force generated was quantified by standard deviation of the force data. Target force error was quantified using root mean square error (RMSE). The onset of a trial was identified by the time point at which the force rose above 10% of the peak force, and the offset of a trial was identified as the time point at which force fell below 10% of the peak force.

Shapiro-Wilk's test of normality was used to determine whether the data were normally distributed, which is one of the key assumptions when implementing ANOVA. Those data that failed the test were subsequently logarithmically transformed for all statistical testing. Differences in mean force as a percent of MVC, variability of force generated, and target force error between each effector were quantified using 1-way ANOVAs with Bonferroni post hoc. Statistical analyses were performed in SPSS version 26 (IBM, Armonk, NY) at a 0.05 alpha level.

Imaging Data Analysis. Imaging data were analyzed with AFNI software (Analysis of Functional NeuroImages; National Institutes of Health, Bethesda, MD), SPM8 software, SUIT toolbox of SPM8, and custom UNIX shell scripts. AFNI software was used to compute whole-brain statistical maps. Cerebellum-specific fine-tuning of the statistical maps was performed with SPM8 and SUIT.

Subject-level Analysis. Each effector (hand, foot, mouth) was analyzed separately. Three functional volumes were collected prior to the beginning of the experiment to allow for magnetization to stabilize, and these volumes were discarded before analysis. Remaining volumes were slice-acquisition-dependent slice-time corrected. The anatomical image was skull stripped using 3dSkullStrip in AFNI. Functional volumes were registered to a base volume via rigid body rotations and aligned with the anatomical image in a single transformation, therefore avoiding repeated image resampling. For the whole-brain analysis, functional volumes were warped into MNI space and smoothed with a 4 mm full-width-half-maximum Gaussian kernel for increasing the signal-to-noise ratio. The blood-oxygen-level-dependent (BOLD) signal in each voxel at every time point was scaled by the mean of its respective time series to normalize the data. The BOLD signal during the 30 s force periods were modeled using a boxcar regressor convolved with the hemodynamic response function. Head motion parameters calculated during the registration step were included in the general linear model as regressors. Head motion between adjacent volumes that was greater than 0.6 mm resulted in the exclusion of both volumes from the regression analysis. For the cerebellum analysis, warping to MNI space and smoothing occurred using the SUIT template after the BOLD signal was normalized and head motion was regressed out of the signal. Head motion above the threshold limit resulted in more than 35% of the functional volumes being excluded from analysis in three individuals. These three subjects were excluded, resulting in 17 participants being included in the final imaging and behavioral analyses. Across the remaining 17 participants, after correcting for head motion, an average of 97.5%, 99.8%, and 89.7% of the volumes remained for the foot, hand, and mouth tasks respectively. Individual-level data were then analyzed using two approaches: group-level voxelwise analysis, and classification analysis.

Group Level Voxelwise Analyses. To statistically compare the BOLD signal for each of the three effectors, activation maps were calculated from the beta coefficients of the whole-brain and the cerebellum using t-tests to compare task (foot, hand, mouth) vs. rest. From the whole-brain t-tests, binary masks were created for each effector, with voxels threshold at p<0.05 and cluster size 2 voxels. These binary masks were then combined in a conjunction analysis to create an overall activation mask, which included voxels that were active in at least one of the hand, foot, or mouth conditions. This activation mask was then applied to the whole-brain t-tests to mask out voxels unrelated to the task. Then, the number of voxels required for a cluster to reach significance when p=0.005 and a=0.05 was calculated using 3dClustSim in AFNI. The cluster extent threshold was 24 voxels. Next, a 1-way ANOVA was implemented on the whole-brain statistical maps to identify significant brain activity that varied as a function of effector (hand vs. foot vs. mouth). A separate ANOVA was run for the SUIT normalized cerebellum data.

Machine Learning Classification Analysis. Supervoxels can be identified using different statistical and machine learning methods such as,but not limited to, a 1-way ANOVA analysis, decision trees, artificial neural networks, and/or support vector machines. A machine learning classification analysis was used to determine whether the BOLD signal could be used to predict which effector was being used and to determine which brain regions have largest contributions to that prediction. Whole-brain maps of beta coefficients for each effector of each subject were used as inputs to the pipeline. Subject-level whole-brain maps were then registered and averaged across effector for each subject 203(a) and then across all subjects 203(b) to create a single whole-brain map. Each voxel in the map represented an average beta coefficient value, which represents the mean percent signal change in the BOLD signal across all subjects, all effectors, and all tasks. Supervoxels were then identified based on a data-driven algorithm 203(c). Supervoxels reduce computational complexity, and by iteratively increasing the number of allowed supervoxels it was possible to determine the relationship between spatial resolution and performance of the classifier.

During classification 206, the supervoxels can be organized into tabular format for machine learning where the supervoxels are represented as feature inputs 206(e). Supervoxels are used as features in the machine learning model 206(f) to produce weights 206(g). Weights are visualized 206(h) to determine anatomical location.

Supervoxel Parcellations. Supervoxels can be generated using an appropriate clustering methodology such as, but not limited to, partitioning methods, hierarchical clustering, fuzzy clustering, density-based clustering, and/or model-based clustering. For example, supervoxels can be generated using a modified simple linear iterative clustering (SLIC) algorithm. The algorithm segments data points with consistent uniformity and is computationally efficient. In the current study, the SLIC algorithm is implemented to segment a voxel map of beta coefficients in three-dimensional space (x, y, z). Other clustering algorithms can also be used. The algorithm takes a single input K, which is the desired number of equally sized supervoxels. The approximate size of each supervoxel is N/K, where N is the number of voxels in the 3-dimensional beta coefficients calculated from fMRI volume. A supervoxel center is found at every grid step

$$S = \sqrt{\frac{N}{K}}$$

for roughly equally sized supervoxels. Clustering begins with an initial seeding of K regularly spaced cluster centers. Each cluster center is then moved to the lowest gradient position within a 3×3×3 neighborhood. The voxel map gradient is computed with:

$$G ( x , y , z ) = {‖ I ( x + 1 , y , z ) - I ( x - 1 , y , z ) ‖}^{2} + {‖ I ( x , y + 1 , z ) - I ( x , y - 1 , z ) ‖}^{2} + {‖ I ( x , y , z + 1 ) - I ( x , y , z - 1 ) ‖}^{2} . \qquad ( 1 )$$

I(x, y, z) is the beta coefficient that corresponds to the voxel position of (x, y, z). ∥ ∥² is the L₂ norm which measures the magnitude of signal difference between two voxels. The gradient accounts for intensity while avoiding placement of centers on edges or noisy voxels. Each voxel in the map is then associated with the nearest cluster center based on a 2S×2S×2S neighborhood around each cluster center. The distance, D, between two voxels i and j is measured using both signal intensities and spatial distances (Equation 2). The compactness variable, m, defines the shape of supervoxels. Higher values of m ensure uniform shapes, while lower values allow supervoxels to adhere to boundaries resulting in irregular shapes. Setting m to a constant value of 0.001 is considered low enough to allow irregular shapes that would be expected given brain anatomy.

$${d}_{intensity} = \sqrt{{( {I}_{i} - {I}_{j} )}^{2}} {d}_{spatial} = \sqrt{{( {x}_{i} - {x}_{j} )}^{2} + {( {y}_{i} - {y}_{j} )}^{2} + {( {z}_{i} - {z}_{j} )}^{2}} D = \sqrt{{( \frac{{d}_{intensity}}{m} )}^{2} + {( \frac{{d}_{spatial}}{s} )}^{2}} \qquad ( 2 )$$

Cluster centers are recomputed using the mean positions of respectively matched voxels. Residual errors are taken using L₁ distance between the old and the new centers. The process of assigning voxels and recomputing centers was repeated until the error was minimized. Connectivity is enforced by relabeling disjointed segments to the largest neighboring cluster. Voxels are then masked using the conjunction map to limit analysis to brain regions that were active during the hand, foot, or mouth task (203(d) of FIG. 2). This may remove supervoxels entirely or remove a portion of the voxels contributing to a supervoxel. This approach guaranteed that the same number of supervoxels was used for each subject in the classification analysis, while also ensuring that supervoxel generation was not biased using between effector contrasts. For each supervoxel, an average beta coefficient was then calculated for each subject across all effectors (203(e) of FIG. 2). Each supervoxel, therefore, represented a single feature, reflecting activity in an area of the brain. Hence, the number of supervoxels indicated how many features were fed to the classification analysis, where an increase in feature number reflects an increase in spatial resolution. The goal of the classification analysis was to determine which supervoxels (i.e., features or brain regions) contributed most to predicting which effector was being used, while also determining overall performance accuracy of the combination of supervoxels. The supervoxel identification process was repeated five times, changing the maximum number of allowed supervoxels from 1 to 10,000 by factors of 10, across iterations. A total of 37 models were generated. Allowed supervoxels describes the input to the SLIC algorithm. However, SLIC does not always generate the input number. Some supervoxels are removed entirely during the algorithm due to minimizing error between supervoxels. This results in a lower number of supervoxels. Generated supervoxels describes the output of the SLIC algorithm. The 10,000 allowed supervoxels generated 1,755 supervoxels which is the largest number of features used among the models, as shown in Table 5 (below). Machine learning was run separately for each iteration that allowed a different number of supervoxels.

For comparison, random uniformly generated supervoxels and pre-existing brain atlases were used to compare against the proposed supervoxel-based parcellation. Each voxel in the random maps were randomly assigned a supervoxel number. The maximum number of random supervoxels ranged from 1 to 1,000, by powers of 10, across iterations totaling 4 different models. These were compared to the generated supervoxels. The automated anatomical labeling (AAL) (n=117 subregions), Brainnetome (n=247 subregions), and Harvard-Oxford cortical atlases (n=68 subregions) were used. These were resampled into 3 mm voxel sizes and a 61×73×61 matrix to match the activity mask.

The supervoxel features were fed into a machine learning algorithm implemented in Python 3.7 to classify effectors (206(f) of FIG. 2). Machine learning can utilize, e.g., gradient boosting decision trees (GBDT) or other appropriate methodology. For example, a modified implementation of gradient boosting decision trees (GBDT), known as extreme gradient boosting (XGBoost), which utilizes an ensemble of weak learners or shallow decision trees can be used. Other forms of machine learning can also be used such as, but not limited to, Decision Tree, Random Forests, Support Vector Machines, Gradient Boosted Tree, Artificial Neural Network, Deep Learning, Bayesian Network, K-Nearest Neighbors, and/or Naive Bayes.

XGBoost Machine Learning Classification. XGBoost (Extreme Gradient Boosting) is an ensemble of gradient boosted decision trees which was implemented to predict effectors using beta coefficient maps as features. A decision-tree uses a tree-like model of sequential and hierarchical decisions. Decision nodes test for a condition and pass the instance through a branch to one of its children nodes based on the condition met by the instance. The tree stops to grow when the nodes cannot be further split, or the stopping criteria is met. The final outcome or leaf determines the class label. Every instance begins at the decision stump or base of the tree. This instance follows a series of decision nodes until it reaches a leaf predicting a class label.

The performance of decision trees can be obtained with an objective function [symbol] ^{m}. This objective function compares the true label and predicted label from the decision trees or base learners. F is a set of ƒ base learners up to M total learners (i.e. the decision trees), where:

$$f = \{ {f}_{1} , {f}_{2} , {f}_{3} , {f}_{4} , ... , {f}_{M} \} . \qquad ( 3 )$$

Prediction scores from individual trees are summed and averaged to get a final prediction label ŷᵢ (Equation 4), where xᵢ is the i^{th} training instance.

$${\hat{y}}_{n} = \frac{1}{m} {\sum }_{m = 1}^{M} {f}_{m} ( {x}_{n} ) \qquad ( 4 )$$

The objective function is calculated with a loss function l comparing the true label yᵢ and predicted label ŷᵢ across N instances. A regularization term Ω(ƒₘ) is added to control model complexity and reduce overfitting of the m^{th} tree of:

$${ℒ}^{m} = {\sum }_{n = 1}^{N} l ( {y}_{n} , {\hat{y}}_{n} ) + Ω ( {f}_{m} ) . \qquad ( 5 )$$

Equation 6 is the logistic regression loss l between the true and predicted label.

$$l = - ( {y}_{n} * log ( {\hat{y}}_{n} ) - ( 1 - {y}_{n} ) * ( log ( 1 - {\hat{y}}_{n} ) ) ) \qquad ( 6 )$$

The regularization term Ω(ƒₘ) tunes the bias and variance trade-off. T is the total number of leaves and wₜ² are the weights from each leaf (Equation 7). Larger weights and larger numbers of leaves are penalized. Terms gamma y and lambda A are tunable in the algorithm. The larger gamma or lambda is, the more conservative the algorithm would be.

$$Ω ( {f}_{m} ) = γ {T}_{t} + \frac{1}{2} λ {\sum }_{t = 1}^{T} {w}_{mt}^{2} \qquad ( 7 )$$

Boosting is an ensemble learning method of sequentially combining weak learners (i.e., shallow decision trees) to form a strong learner. Each sequential tree aims to reduce the error of the previous tree. The ƒ^{th} tree in boosting is chosen from a forest of decision trees. A forest is generated with a fixed number of decision trees. XGBoost determines the maximum number of trees as each new tree is sequentially tested. The ƒ^{th} tree is selected as the best performing tree in the forest. Equation 8 is the updated prediction label that takes the result from the previous tree, where t is the iteration in boosting.

$${{\hat{y}}_{n}}^{( t )} = {\sum }_{m = 1}^{M} {f}_{m} ( {x}_{n} ) = {{\hat{y}}_{n}}^{( t - 1 )} + {f}_{t} ( {x}_{n} ) \qquad ( 8 )$$

Equation 9 is the updated objective function for boosted decision trees. This equation substitutes Equation 8 (ŷᵢ) into Equation 5. Now the current model considers the results from the previous model.

$${ℒ}^{m} = {\sum }_{n = 1}^{N} l ( {y}_{n} , {{\hat{y}}_{n}}^{( t - 1 )} + {f}_{m} ( {x}_{n} ) ) + Ω ( {f}_{m} ) \qquad ( 9 )$$

The gradient in XGBoost describes the method of minimizing the loss function with gradient descent. The direction of gradient descent is approximated with a 2^{nd} order Taylor series expansion as:

$$f ( x ) \approx f ( a ) + {f}^{′} ( a ) ( x - a ) + \frac{1}{2} {f}^{″} ( a ) {( x - a )}^{2} . \qquad ( 10 )$$

Here, a=ŷₙ^{(t-1)}), h=ƒₘ(xₙ), and ƒ(a)=l(yₙ,ŷₙ^{(t-1)}). These are substituted into the Taylor series approximation as:

$${ℒ}^{m} = {\sum }_{n = 1}^{N} l ( {y}_{n} , {{\hat{y}}_{n}}^{( t - 1 )} ) + ( \frac{\partial l ( {y}_{n} , {{\hat{y}}_{n}}^{( t - 1 )} )}{\partial {{\hat{y}}_{n}}^{( t - 1 )}} ) {f}_{m} ( {x}_{n} ) + ( \frac{{\partial }^{2} l ( {y}_{n} , {{\hat{y}}_{n}}^{( t - 1 )} )}{\partial {{\hat{y}}_{n}}^{{( t - 1 )}^{2}}} ) {{f}_{m} ( {x}_{n} )}^{2} + Ω ( {f}_{m} ) . \qquad ( 11 )$$

Equation 12 simplifies the expansion where

$${g}_{i} = ( \frac{\partial l ( {y}_{n} , {{\hat{y}}_{n}}^{( t - 1 )} )}{\partial {{\hat{y}}_{n}}^{( t - 1 )}} ) and {h}_{i} = ( \frac{{\partial }^{2} l ( {y}_{n} , {{\hat{y}}_{n}}^{( t - 1 )} )}{\partial {{\hat{y}}_{n}}^{{( t - 1 )}^{2}}} ) .$$

The final objective function (Equation 12) describes the gradient boosting decision trees.

$${ℒ}^{m} = {\sum }_{n = 1}^{N} ( ( l ( {y}_{n} , {{\hat{y}}_{n}}^{( t - 1 )} ) + {g}_{n} {f}_{m} ( {x}_{n} ) + \frac{1}{2} {h}_{n} {{f}_{m}}^{2} ( {x}_{n} ) ) + Ω ( {f}_{m} ) \qquad ( 12 )$$

Traditional gradient descent minimizes a cost function ƒ(x) by following the negative gradient of the function at each step. However, the exact minimum is not found after a single step. Convergence can be quite slow and large steps can overshoot the minimum. The 2^{nd} order approximation gives more information in the direction of the minimum. A tree that is evaluated with a full cost function l, has a computation every new split. XGBoost is quicker since the computations are a lot less. l(yₙ,ŷₙ^{(t-1)}), gᵢ, and hᵢ are constants in the approximation and are computed once and reused for all decision tree splits. ƒₘ(xₙ) and Ω(ƒₘ) remain to be calculated each split.

XGBoost enhances the gradient boosting implementation with the inclusion of additional features. These include but are not limited to hardware optimization, parallelized tree building, L1 and L2 regularization, and handling of missing data. XGBoost yields improved model performance and parallelized computational speed compared to other decision tree-based models. This is ideal in comparing multiple models with many features. The algorithm outputs weights for supervoxels (206(g) of FIG. 2), providing information on which supervoxels (and therefore brain areas) are identified as the most important when classifying effectors (206(h) of FIG. 2).

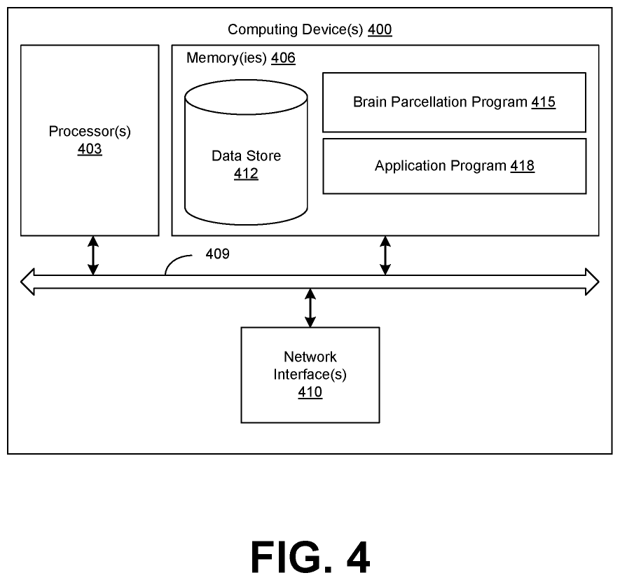

A nested 5-fold cross-validation (CV) can be used to estimate the unbiased accuracy of each iteration of supervoxel combinations. A traditional training (80% of the data) and testing (20% of the data) split can bias the results depending on the choice of the test set. Nested 5-fold CV provides a more unbiased performance measure. Nested 5-fold CV divides the data into five folds or groups of subjects with an equal number of subjects per fold. Each group is stratified to retain an equal ratio from each effector (hand, foot, and mouth). Nested CV uses an outer loop to estimate the error, while the inner loop is used for model selection and to tune hyperparameters. A single iteration of CV uses a single fold for testing and the rest for training. Each loop rotates the training and test folds until all folds have been used for testing once. In the example of FIG. 3, the full dataset (a) includes a total of 51 task-based data sets or beta coefficient voxel maps across 17 subjects. Each subject has a hand, foot, and mouth voxel map. The full dataset is split and stratified into 5-folds where each fold has 10-11 voxel maps and roughly the same amount of each task. As shown in (b) of FIG. 3, each fold is iteratively used as a test fold where the rest of the data is used for training. The cross validation outer loop controls error estimation and provides an overall performance measure from this dataset. In (c) of FIG. 3, the cross validation inner loop performs cross validation using the training subsets from the outer loop to tune hyperparameters. The nested cross validations estimate an unbiased performance of a model.

Performance of the models and hyperparameters are recorded each loop iteration. A broad parameter dictionary can be used to account for the wide range of supervoxels (i.e., 1-10,000) (Table 1). A randomized search of parameters using different combinations was used to determine the ideal parameters in optimizing performance. The parameters with the best classification performance were used in the final model.

TABLE 1
List of the parameters and values considered. A randomized
search was performed to sample multiple combinations
of parameters to optimize performance.

| Parameters | Value |
|---|---|
| Learning_rate | 0.01, 0.05, 0.10, 0.20, 0.30, 0.40 |
| n_estimators | 50, 100, 500, 1000 |
| max_depth | 5, 10, 15, 20, 25 |
| subsample | 0.5, 0.6, 0.7, 0.8, 0.9, 1.0 |
| colsample_bytree | 0.5, 0.6, 0.7, 0.8, 0.9, 1.0 |
| scale_pos_weight | 30, 40, 50, 300, 400, 500, 600, 700 |
| gamma | 0.00, 0.05, 0.10, 0.15, 0.20, 1, 5 |

Separate and independent final models for each supervoxel model are created to produce weights. Each model is trained on the entire dataset. Results and parameters from CV are only used to estimate performance and are not used in the final model to avoid bias and overfitting. Each supervoxel model produces weights for every feature used in the model. These weights represent the importance of a supervoxel in determining an effector. The centroid locations, volume, and values are recorded.

Classification Evaluation Metrics. In the classification analysis, a modified SLIC algorithm can be used to automatically segment task-based fMRI using supervoxels. Supervoxels computationally group regions based on voxel similarities alone. Multiple models of generated supervoxels were compared in classifying different effectors (hand, foot, and mouth). Increasing the number of supervoxels also increases the spatial resolution of brain parcellation. Task-based fMRI was utilized from 17 subjects, each with hand, foot, and mouth fMRI data yielding 51 total data sets. Classification used nested 5-fold CV for each supervoxel model to estimate the performance. An additional and separate model is generated from each supervoxel group using all 51 data sets to produce the weights. To compare the performances between models, the balanced accuracy, macro-precision, macro-recall, macro-F1 score, and confusion matrices were calculated for any given model.

Balanced accuracy is the mean of Sensitivity and Specificity, which is more appropriate for unbalanced data, i.e., data with unbalanced number of samples for each label. Here sensitivity is the same as recall defined below, and specificity (a.k.a. true negative rate) is the ratio of negative samples correctly predicted among the total number of negative samples. This is because even though there was an equal number of labels for hand, foot, and mouth, stratified 5-fold CV still has slightly unbalanced data in each fold because there are 51 samples in total.

$${Accuracy}_{B} = \frac{Sensitivity + Specificity}{2} \qquad ( 13 a )$$

Precision is the fraction of examples predicted to be a certain label that actually belong to that label (Equation 13b). For example, the machine learning model predicts 50 samples to be Foot and among them, only 40 are actually Foot samples. Then Precision equals to 40/50=80%. A precision score is calculated for each label, then averaged to obtain a macro-precision score

$${Precision}_{l ∈ \{ Hand , Foot , Mouth \}} = \frac{Correctly Predicted \# of Label l}{Predicted \# of Label l} \qquad ( 13 b )$$

Recall (a.k.a. Sensitivity) measures the fraction of samples belonging to a certain label that are correctly predicted (Equation 13c). For example, among 60 samples of label Hand, 30 samples are predicted as Hand. Then recall equals 30/60=50%. One recall score is calculated for each label. The recall score is averaged across all labels to give a macro-recall score.

$${Recall}_{l ∈ \{ Hand , Foot , Mouth \}} = \frac{Correctly Predicted \# of Samples with Label l}{True \# of Samples with Label l} \qquad ( 13 c )$$

The F1 score takes the harmonic mean (Equation 13d) of precision and recall of each label, then these F1 scores are averaged to obtain a macro-F1 score across labels (Equation 13d). F1 score conveys the balance between precision and recall.

$$F 1 Score = 2 * \frac{Precision * Recall}{Precision + Recall} \qquad ( 13 d )$$

Lastly, confusion matrices are generated for each supervoxel model to show the distribution of predicted and true labels. A 3-by-3 confusion matrix is generated where each row and column correspond to the Hand, Foot, and Mouth labels. The rows contain the true labels whereas the columns contain the predicted labels, showing the number of different types of predictions. Labels that are correctly predicted (predicting a hand label to be a hand) lie upon the main diagonal. All other values in the confusion matrix represent number of misclassified samples. All previously mentioned metrics can be calculated from the confusion matrix.

Occlusion Tests. An occlusion test is used to show the change in the performance of a model by occluding a specific brain region (e.g. set values to zeros). The larger the contribution a brain region has in the classification, the larger the performance degradation in the occluded model. To evaluate the contribution of a certain brain region(s), the values of the brain region(s) are set to zero for all data (both training and test data). Then use the same machine learning can be used to build a model using data where the region has been lesioned and compute the classification performance on the test data. The change in the classification performance reflect the importance of the corresponding brain region(s). In this work, the AAL atlas was used to occlude specific brain regions and set the supervoxel number to 5000 when creating the adaptive supervoxel-based parcellation, as a good balance between the spatial resolution and computation time, leading to 1038 supervoxels in the actual parcellation model.

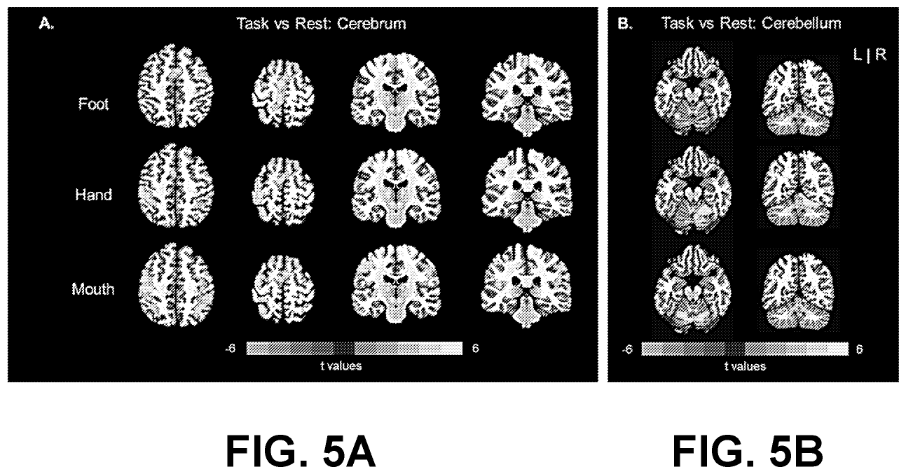

System Implementation. With reference to FIG. 4, shown is a schematic block diagram of a computing device 400 that can be utilized for dynamic brain parcellation. In some embodiments, among others, the computing device 400 may represent a mobile device (e.g., a smartphone, tablet, computer, etc.). Each computing device 400 includes at least one processor circuit, for example, having a processor 403 and a memory 406, both of which are coupled to a local interface 409. To this end, each computing device 400 may comprise, for example, at least one server computer or like device. The local interface 409 may comprise, for example, a data bus with an accompanying address/control bus or other bus structure as can be appreciated.

In some embodiments, the computing device 400 can include one or more network interfaces 410. The network interface 410 may comprise, for example, a wireless transmitter, a wireless transceiver, and a wireless receiver. As discussed above, the network interface 410 can communicate to a remote computing device using a Bluetooth protocol. As one skilled in the art can appreciate, other wireless protocols may be used in the various embodiments of the present disclosure.

Stored in the memory 406 are both data and several components that are executable by the processor 403. In particular, stored in the memory 406 and executable by the processor 403 are a brain parcellation program 415, application program 418, and potentially other applications. Also stored in the memory 406 may be a data store 412 and other data. In addition, an operating system may be stored in the memory 406 and executable by the processor 403.

It is understood that there may be other applications that are stored in the memory 406 and are executable by the processor 403 as can be appreciated. Where any component discussed herein is implemented in the form of software, any one of a number of programming languages may be employed such as, for example, C, C++, C#, Objective C, Java®, JavaScript®, Perl, PHP, Visual Basic®, Python®, Ruby, Flash®, or other programming languages.

A number of software components are stored in the memory 406 and are executable by the processor 403. In this respect, the term “executable” means a program file that is in a form that can ultimately be run by the processor 403. Examples of executable programs may be, for example, a compiled program that can be translated into machine code in a format that can be loaded into a random access portion of the memory 406 and run by the processor 403, source code that may be expressed in proper format such as object code that is capable of being loaded into a random access portion of the memory 406 and executed by the processor 403, or source code that may be interpreted by another executable program to generate instructions in a random access portion of the memory 406 to be executed by the processor 403, etc. An executable program may be stored in any portion or component of the memory 406 including, for example, random access memory (RAM), read-only memory (ROM), hard drive, solid-state drive, USB flash drive, memory card, optical disc such as compact disc (CD) or digital versatile disc (DVD), floppy disk, magnetic tape, or other memory components.

The memory 406 is defined herein as including both volatile and nonvolatile memory and data storage components. Volatile components are those that do not retain data values upon loss of power. Nonvolatile components are those that retain data upon a loss of power. Thus, the memory 406 may comprise, for example, random access memory (RAM), read-only memory (ROM), hard disk drives, solid-state drives, USB flash drives, memory cards accessed via a memory card reader, floppy disks accessed via an associated floppy disk drive, optical discs accessed via an optical disc drive, magnetic tapes accessed via an appropriate tape drive, and/or other memory components, or a combination of any two or more of these memory components. In addition, the RAM may comprise, for example, static random access memory (SRAM), dynamic random access memory (DRAM), or magnetic random access memory (MRAM) and other such devices. The ROM may comprise, for example, a programmable read-only memory (PROM), an erasable programmable read-only memory (EPROM), an electrically erasable programmable read-only memory (EEPROM), or other like memory device.

Also, the processor 403 may represent multiple processors 403 and/or multiple processor cores and the memory 406 may represent multiple memories 406 that operate in parallel processing circuits, respectively. In such a case, the local interface 409 may be an appropriate network that facilitates communication between any two of the multiple processors 403, between any processor 403 and any of the memories 406, or between any two of the memories 406, etc. The local interface 409 may comprise additional systems designed to coordinate this communication, including, for example, performing load balancing. The processor 403 may be of electrical or of some other available construction.

Although the brain parcellation program 415 and the application program 418, and other various systems described herein may be embodied in software or code executed by general purpose hardware as discussed above, as an alternative the same may also be embodied in dedicated hardware or a combination of software/general purpose hardware and dedicated hardware. If embodied in dedicated hardware, each can be implemented as a circuit or state machine that employs any one of or a combination of a number of technologies. These technologies may include, but are not limited to, discrete logic circuits having logic gates for implementing various logic functions upon an application of one or more data signals, application specific integrated circuits (ASICs) having appropriate logic gates, field-programmable gate arrays (FPGAs), or other components, etc. Such technologies are generally well known by those skilled in the art and, consequently, are not described in detail herein.

Also, any logic or application described herein, including the brain parcellation program 415 and the application program 418, that comprises software or code can be embodied in any non-transitory computer-readable medium for use by or in connection with an instruction execution system such as, for example, a processor 403 in a computer system or other system. In this sense, the logic may comprise, for example, statements including instructions and declarations that can be fetched from the computer-readable medium and executed by the instruction execution system. In the context of the present disclosure, a “computer-readable medium” can be any medium that can contain, store, or maintain the logic or application described herein for use by or in connection with the instruction execution system.

The computer-readable medium can comprise any one of many physical media such as, for example, magnetic, optical, or semiconductor media. More specific examples of a suitable computer-readable medium would include, but are not limited to, magnetic tapes, magnetic floppy diskettes, magnetic hard drives, memory cards, solid-state drives, USB flash drives, or optical discs. Also, the computer-readable medium may be a random access memory (RAM) including, for example, static random access memory (SRAM) and dynamic random access memory (DRAM), or magnetic random access memory (MRAM). In addition, the computer-readable medium may be a read-only memory (ROM), a programmable read-only memory (PROM), an erasable programmable read-only memory (EPROM), an electrically erasable programmable read-only memory (EEPROM), or other type of memory device.

Further, any logic or application described herein, including the brain parcellation program 415 and the application program 418, may be implemented and structured in a variety of ways. For example, one or more applications described may be implemented as modules or components of a single application. Further, one or more applications described herein may be executed in shared or separate computing devices or a combination thereof. For example, a plurality of the applications described herein may execute in the same computing device 400, or in multiple computing devices in the same computing environment. Additionally, it is understood that terms such as “application,” “service,” “system,” “engine,” “module,” and so on may be interchangeable and are not intended to be limiting.

### Results

Force Data. Table 2 shows the mean±SD of all behavioral variables assessed. No significant difference was found between effectors for the mean force generated as a percentage of MVC (F(2,48)=2.7; p=0.079), or for the target force error (F(2,48)=0.4; p=0.661). A significant effect of effectors was found for the variability of force generated (F(2,48)=43.1; p<0.001). A Bonferroni post hoc revealed that the variability of force generated by the hand (0.40±0.09; p<0.001) and foot (0.62±0.44; p<0.001) was greater than the variability of force generated by the mouth (0.15±0.11). No significant difference was found between the variability of force generated by the hand and foot (p=0.069).

TABLE 2
Mean ± SD of force, force variability, and target force error
for the hand, foot, and mouth effector.

| | | Force Variability | Target Force Error |
|---|---|---|---|
| Effector | Mean Force | (SD) | (RMSE) |
| Hand | 9.33 ± 0.64 | 0.40 ± 0.09 | 0.74 ± 0.70 |
| Foot | 9.53 ± 0.97 | 0.62 ± 0.44 | 0.70 ± 0.28 |
| Mouth | 9.86 ± 0.34 | 0.15 ± 0.11 | 0.59 ± 0.16 |

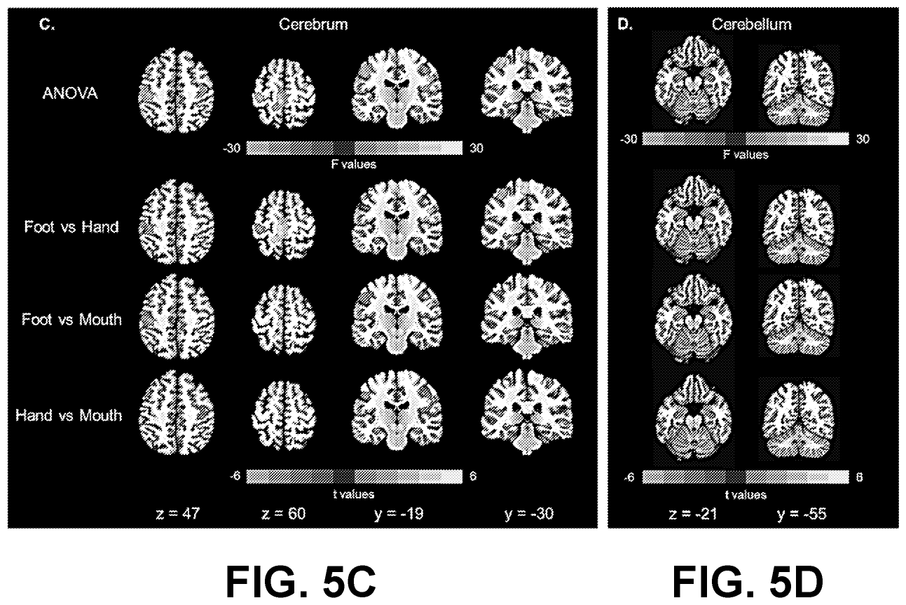

Brain Imaging Data. FIG. 5A shows activation maps (task vs rest) for each of the three effectors based on the whole-brain analysis (p=0.005; a=0.05; cluster size=24 voxels). In FIG. 5A, whole brain activation maps show t-values for each of the three effectors vs. rest. All three effectors show activation of the supplementary motor area. The foot (top) shows activation in the medial sensorimotor cortex. The hand (middle) shows activation laterally in the left hemisphere, consistent with the right hand being used. The mouth (bottom) shows more widespread, lateral activation bilaterally. Qualitatively, the well-established somatotopic organization of the foot, hand, and mouth areas of the sensorimotor cortex can be seen. For the foot, activation was localized medially in the paracentral lobule, consistent with the foot and leg area of the sensorimotor cortex. Hand activation was localized laterally in the left hemisphere, consistent with the right hand being used to perform the task. Finally, the mouth task resulted in more lateral and inferior activation in the left hemisphere when compared to the hand, as well as more widespread and bilateral activity.

FIG. 5B shows cerebellar activation maps for each of the three effectors (p=0.005; a=0.05; cluster size=24 voxels). In FIG. 5B, cerebellum activation maps show t-values for each of the three effectors vs. rest. The foot (top) shows activity in the right anterior lobules, as well as bilateral activity in the posterior lobules of the cerebellum. The hand (middle) activity is localized to the right anterior and posterior lobules. The mouth (bottom) shows bilateral activation of the posterior lobules. For the foot, the cerebellum activity was localized to right anterior lobules IV-V, as well as bilaterally in posterior lobule VI. The majority of activity during the hand task was localized to the ipsilateral right anterior and posterior lobule VI. Finally, the mouth task resulted in bilateral activation in posterior lobule VI.

FIG. 5C shows results of the separate 1-way ANOVAs used to identify areas that significantly differed between effector. FIG. 5C illustrates whole brain 1-way ANOVA (top) identifying areas that significantly differed between the three effectors (foot, hand, mouth), and follow-up t-tests (bottom) determined which effector(s) differed at each region. Follow-up t-tests (rows 2, 3, and 4) show which effector(s) differed within each region identified in the ANOVA. For example, the Foot vs Hand follow-up t-test shows significantly greater activation for the foot in the medial sensorimotor cortex, which were indicated by the warm colors (positive F values). This medial paracentral lobule region is consistent with prior work localizing peak BOLD responses during ankle movement to MNI coordinates of −7, −38, 74 and −7, −35, 73 (after conversion of coordinates to MNI space). Significantly greater activation for the hand was identified laterally in the left sensorimotor cortex, indicated by the cool colors (negative F values), and is consistent with hand movement experiments localizing peak BOLD responses to MNI (−38, −24, 56). Similarly, the Foot vs Mouth follow-up t-test shows significantly greater activation for the foot in the medial sensorimotor cortex, while significantly greater activation for the mouth effector was more lateral, aligning with localization of tongue movement in the left hemisphere (−54, −5, 30; after conversion to MNI space). The bottom row shows the Hand vs Mouth comparison revealed significantly greater bilateral activation for the mouth.

In the cerebellum, FIG. 5D shows significantly greater activation for the foot in right lobule IV-V, while the hand effector shows significantly greater activation in right lobule VI. FIG. 5D illustrates cerebellum 1-way ANOVA (top) identifying areas that significantly differed between the three effectors (foot, hand, mouth). Follow-up t-tests (bottom) determined which effector(s) differed at each region. Images were threshold at p=0.005, a=0.05, and cluster size=24 voxels. Greater activation for the mouth as compared to the foot was evident in left lobule VI. Finally, the cerebellar Hand vs Mouth comparison shows significantly greater activation for the hand effector in right lobule VI, while the mouth effector shows significantly greater activation in left lobule VI. Overall, the results showed that 11 whole-brain and 3 cerebellar regions significantly differed between the effectors (p<0.005; a=0.05; cluster size=24 voxels). These regions, along with the t-value for each of the three effector comparisons, are shown in Table 3.

TABLE 3

| Cluster | MNI | | | | |
|---|---|---|---|---|---|
| Size | Coordinates | Anatomical | F vs H | F vs M | H vs M |
| (voxels) | (x, y, z) | Location | (t-value) | (t-value) | (t-value) |
| 678 | −48, −6, 39 | L Postcentral Gyrus | 7.26 | 2.51 | 5.14 |
| | | (Hand area) | (F < H) | (F < M) | (H > M) |
| 430 | −6, −32, 67 | L Paracentral Lobule | 7.58 | 5.07 | 2.71 |
| | | (Leg area) | (F > H) | (F > M) | (H < M) |
| 126 | −35, −67, 30 | L Middle | 1.06 | 3.62 | 5.36 |
| | | Occipital Gyrus | (F > H) | (F < M) | (H < M) |
| 121 | 48, −20, 13 | R Rolandic | 4.48 | 0.64 | 4.96 |
| | | Operculum | (F > H) | (F < M) | (H < M) |
| 111 | 46, −61, 34 | R Angular Gyrus | 2.31 | 4.11 | 2.81 |
| | | | (F < H) | (F < M) | (H < M) |
| 85 | 45, −16, 43 | R Precentral Gyrus | 2.02 | 2.76 | 5.23 |
| | | | (F > H) | (F < M) | (H < M) |
| 45 | 21, −77, 32 | R Superior | 0.55 | 4.01 | 4.94 |
| | | Occipital Gyrus | (F > H) | (F < M) | (H < M) |
| 36 | −46, −28, 9 | L Superior | 1.95 | 3.66 | 5.61 |
| | | Temporal Gyrus | (F > H) | (F < M) | (H < M) |
| 36 | −41, −72, 15 | L Middle | 1.72 | 2.61 | 5.05 |
| | | Occipital Gyrus | (F > H) | (F < M) | (H < M) |
| 26 | −38, −18, 0 | L Insula Lobe | 1.22 | 3.36 | 4.44 |
| | | | (F > H) | (F < M) | (H < M) |
| 25 | 44, 1, −34 | R Inferior | 2.65 | 2.25 | 5.43 |
| | | Temporal Gyrus | (F < H) | (F > M) | (H > M) |
| 82 | 17, −56, −20 | R Cerebellum | 8.42 | 3.14 | 4.44 |
| | | Lobules IV-V | (F < H) | (F < M) | (H > M) |
| 53 | −14, −59, | L Cerebellum | 1.41 | 5.56 | 4.31 |
| | −20 | Lobule VI | (F < H) | (F < M) | (H < M) |
| 27 | 27, −48, −38 | R Cerebellum | 0.66 | 4.18 | 3.52 |
| | | Lobule VI | (F < H) | (F < M) | (H < M) |

Clusters showing a significant (p = 0.005; α = 0.05; cluster size = 24 voxels) effect of effector based on ANOVA and clusters showing between effector differences for each t-test.
MNI coordinates indicate the cluster center of mass.
T-values for each effector comparison are taken from the cluster’s peak voxel location.
Significant t-values are highlighted with bold text.

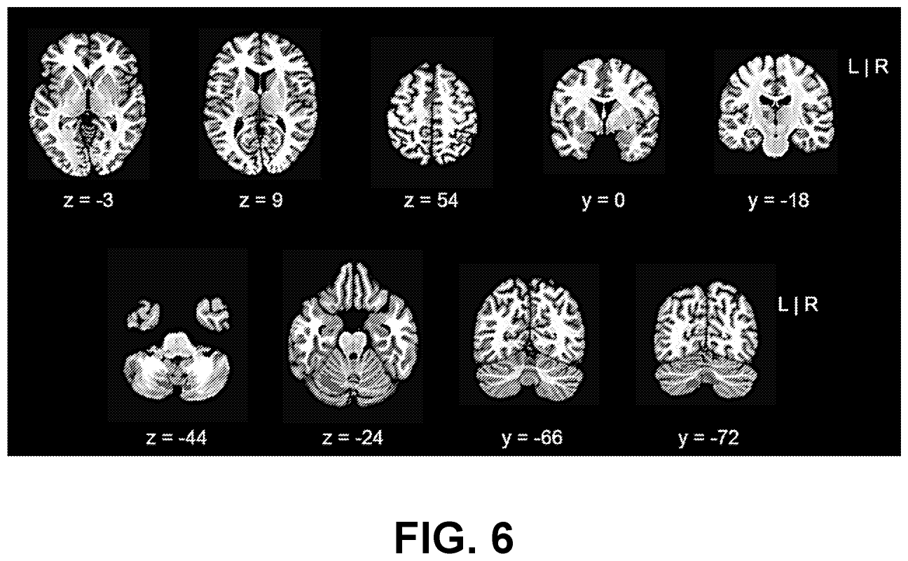

Common areas of activation across the three effectors are shown in FIG. 6. In FIG. 6, whole brain (top) and cerebellum (bottom) overlap maps show common areas of activation across all three effectors. The images were threshold at p=0.005, a=0.05, and cluster size=24 voxels.These areas are consistent with the visuomotor network, and include the supplementary motor area, right and left middle occipital gyrus, right and left inferior parietal lobule, left putamen, left thalamus, left cerebellum lobules VI and VIII, and right cerebellum lobule VII (Table 4).

TABLE 4
Overlap of activity across all three effectors (foot, hand, and
mouth) showing common areas of activation.

| Cluster Size | MNI Coordinates | |
|---|---|---|
| (voxels) | (x, y, z) | Anatomical Location |
| 196 | 1, −1, 61 | Supplementary Motor Area |
| 83 | 33, −88, −3 | Right Inferior Occipital Gyrus |
| 79 | −26, 2, −1 | Left Putamen |
| 56 | −30, −90, −4 | Left Middle Occipital Gyrus |
| 35 | 34, −50, 53 | Right Inferior Parietal Lobule |
| 27 | −34, −53, 58 | Left Superior Parietal Lobule |
| 24 | −13, −19, 6 | Left Thalamus |
| 66 | −25, −65, −26 | Left Cerebellum Lobule VI |
| 44 | −14, −72, −45 | Left Cerebellum Lobule VIII |
| 32 | 6, −74, −40 | Right Cerebellum Lobule VII |

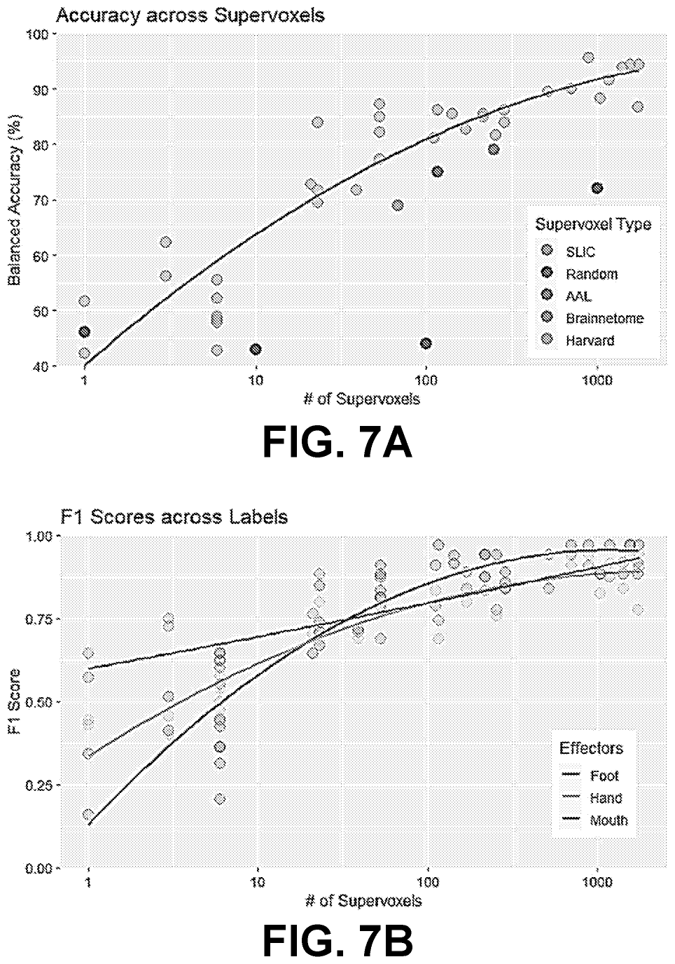

Classification Analysis. Table 5 shows the performance of a subset of different models, including balanced accuracy, macro-precision, macro-recall, and macro-F1 score. Across all of these measures, the numbers increase with an increase in the number of supervoxels. For instance, balanced accuracy increased from 0.42 to 0.94 from 1 supervoxel to 1,755 supervoxels. As the number of allowed features increased, the model is better able to predict which effector was being used. This pattern in accuracy is also shown in FIG. 7A across all supervoxel models and indicates that performance surpasses 80% at around 100 supervoxels. FIG. 7A illustrates accuracy scores across supervoxels. An increase in balanced accuracy is seen with an increase in the number of generated supervoxels. Generated supervoxels are the outputs of the SLIC algorithm. The number of supervoxels are on a logarithmic scale. A 2^{nd} degree polynomial trend line was fit to show the steady increase from 1 to 1755 supervoxels. Randomly generated supervoxels and brain atlases have lower accuracy than the segmented supervoxels. All models past 1000 supervoxels are in the 85-95% accuracy range. FIG. 7A also shows that random supervoxels (n<100) performed worse than random guessing and overall had accuracy under the average segmented supervoxel accuracies. Furthermore, AAL, Brainnetome, and Harvard-Oxford cortical atlases also yield lower accuracies than the SLIC supervoxel accuracies.

Table 5 and FIG. 7B show macro-F1 scores (along with macro-precision and macro-recall), which account for the performance across effector labels. FIG. 7B illustrates F1 scores across labels. F1 scores increase at different rates across effector. The F1 scores focus on the misclassification of effectors, and show the effectors perform differently. The foot effector has the worst performance when a lower number of supervoxels is used (i.e., less spatial resolution). F1 scores greatly increase until around 1000 supervoxels where the foot scores begin to plateau. All three effectors benefit from an increased number of supervoxels or greater spatial resolution. The macro-F1 scores are in line with the balanced accuracy scores and increase as the number of allowable features increased. Together these data show that the supervoxel models do well with differentiating effectors with increased spatial resolution.

TABLE 5
Evaluation metrics. Accuracy and macro-F1 score increase
as the number of supervoxels increases. The maximum
number of supervoxels represents the number input
into the SLIC algorithm, whereas the number
of actual supervoxels represent the actual
supervoxels used for classification. Accuracy,
precision, recall, and F1 score show an
increase with an increase in spatial resolution
(i.e., number of supervoxels). The F1 score
paired with the accuracy indicates the model did not
have any major issues with misclassified labels.

| Max # of | # of Actual | Balanced | Macro | Macro | Macro |
|---|---|---|---|---|---|
| Supervoxels | Supervoxels | Accuracy | Precision | Recall | F1 Score |
| 1 | 1 | 0.42 | 0.37 | 0.42 | 0.38 |
| 10 | 6 | 0.49 | 0.48 | 0.49 | 0.46 |
| 100 | 53 | 0.85 | 0.90 | 0.85 | 0.86 |
| 1000 | 285 | 0.84 | 0.89 | 0.84 | 0.84 |
| 10000 | 1755 | 0.94 | 0.95 | 0.94 | 0.94 |

The individual F1 score for each effector in each model shows different trends in classification complexity (FIG. 7B). Closer inspection shows the difficulty of classifying foot effector maps when lower numbers of supervoxels are used (i.e., lower spatial resolution). This suggests the foot requires greater spatial resolution to better classify. The performance improvement in classifying the mouth effector is not as steep as the foot effector, indicating that the mouth effector is more distinctive in the fMRI data even at relatively lower spatial resolution in the brain parcellation. The hand effector maps fall in between the other two effectors and follow a similar trend to the foot effector maps. Additionally, the scores of all three labels meet around 50 supervoxels suggesting that this is a minimum resolution to classify between the three effectors.

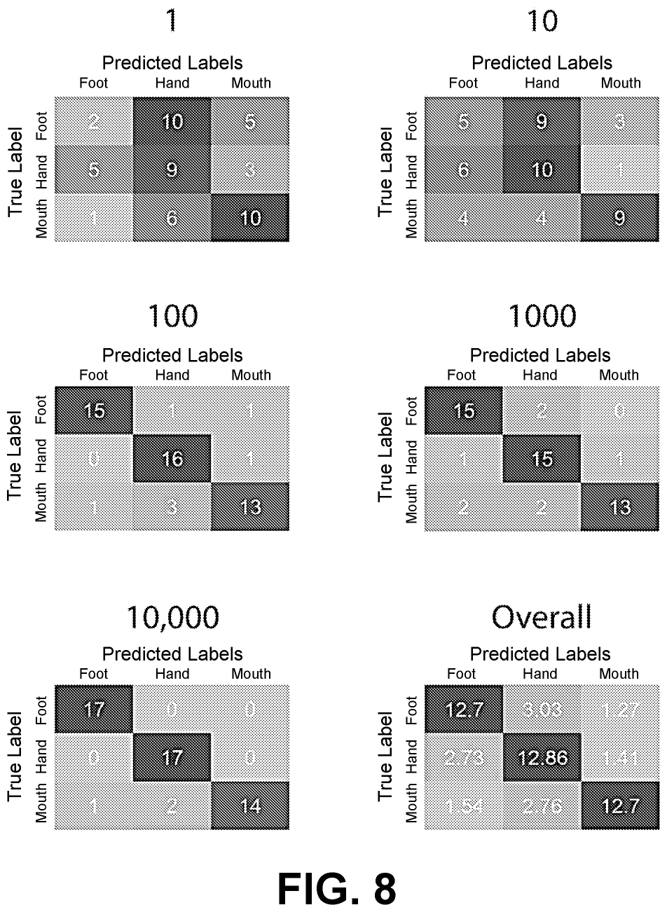

Confusion matrices show the prediction relationships between true effector labels and predicted effector labels (FIG. 8). In FIG. 8, the confusion matrices show different supervoxel models from 1 to 10,000. In each matrix, the main diagonal represents a correct prediction. Smaller supervoxel numbers tend to reflect the lower accuracies and metrics with a seemingly normal distribution of misclassifications. All confusion matrices were averaged together to generate an overall model. The 1 supervoxel model which showed poor accuracy in a seemingly random distribution in the confusion matrix because only one supervoxel is used, which is the entire brain conjunction map as one region. As the supervoxels increase from 1 to 10,000 the main diagonal scores become higher with less misclassification across labels. The misclassifications are fairly distributed as supervoxels increase with the exception of the 10,000 supervoxel model. Three mouth labels were incorrectly classified in the 10,000 supervoxel confusion matrix. The distribution of the misclassifications could simply be a result of noise from the single model. Lastly, an overall confusion matrix was averaged from all confusion matrices to determine if there were difficult labels across resolutions. The overall model shows an equal distribution of correctly predicted labels. There is generally at least one misclassified label and no majorly difficult labels seen across all models. The matrix suggests that all models performed equally across all three labels with nearly even scores across the main diagonal.

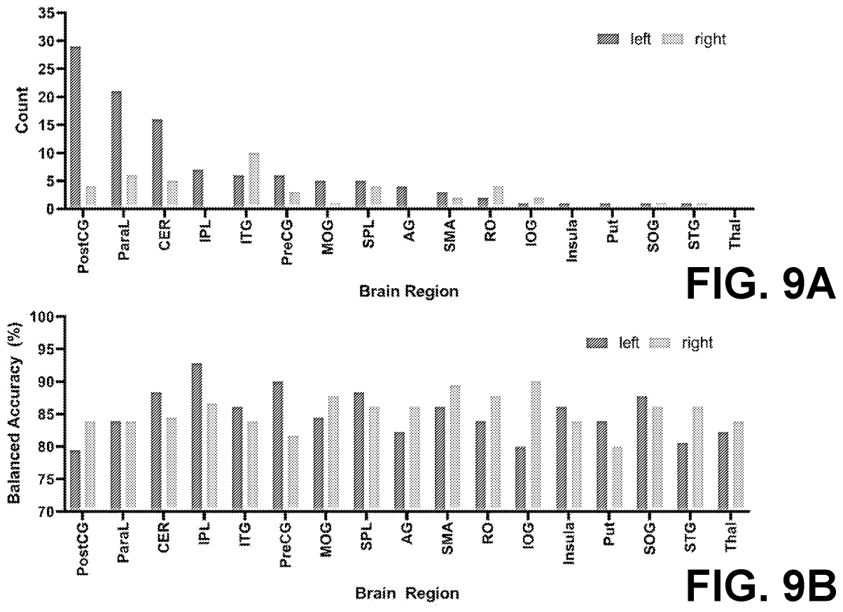

The top 10 supervoxels with the highest weights for classifying effector were identified for each model and localized based on the supervoxel's center of mass. This localization was performed for each of the models from 30 to 10,000 specified supervoxels (a total of 26 models), as the models below 30 specified supervoxels produced less than 10 active supervoxels. Next, how many times a region was identified across the models was counted. The full list of regions identified by the classification analysis is listed in Table 6, with a subset of these regions shown in FIG. 9A. In FIG. 9A, a subset of regions identified by the classification analysis and the number of times the region was identified for the right and left hemisphere. The majority of regions were localized to the left hemisphere consistent with movement of the right effector. The left (contralateral) cerebellum was identified more often than the expected right (ipsilateral) cerebellum, likely because the classification algorithm could more easily exploit the bilateral activity associated with the mouth task. Note that the models can potentially identify multiple subregions within one larger region (e.g., multiple supervoxels in postcentral gyrus) such that the count can be higher than the number of models tested. FIG. 9A shows that supervoxels were most often localized to the postcentral gyrus (n=29), paracentral lobule (n=21), and cerebellum (n=16). The majority of regions were localized to the left hemisphere consistent with movement of the right effector. An occlusion paradigm was next used to determine redundancy in somatotopy, but occluding each individual region and then re-running the model to see its effect on balanced accuracy. In FIG. 9B, each identified brain region was independently removed or occluded from the classification analysis. Regions were removed at the same time the conjunction map is applied. Each model uses a pre-specified 5000 supervoxels, leading to a parcellation model of 1038 regions. The accuracy shows the performance of a model without the respective brain region. Lower accuracies indicate the relative importance of a region in classification. Although FIG. 9B shows that removing the left post-central gyrus resulted in accuracy dropping below 80%, in general the Figure shows that accuracy remained in the 80-90% range, indicating that no one area is essential in predicting effector.

TABLE 6
(Supplementary Table). List of all regions identified by
the classification analysis, as well as the number of times
the region was identified by the different models.

| | Classification | | Effector- | | Effector- | |
|---|---|---|---|---|---|---|
| | # of occurrences | | dependent | | Independent | |
| | L | R | L | R | L | R |
| Postcentral Gyrus | 29 | 4 | X | | | |
| Paracentral Lobule | 21 | 6 | X | | | |
| Cerebellum | 16 | 5 | X | X | X | X |
| Precuneus | 9 | 3 | | | | |
| Inferior Parietal Lobule | 7 | 0 | | | | X |
| Middle Frontal Gyrus | 7 | 4 | X | | | |
| Inferior Temporal Gyrus | 6 | 10 | | X | | |
| Precentral Gyrus | 6 | 3 | | X | | |
| Fusiform Gyrus | 5 | 2 | | | | |
| Lingual Gyrus | 5 | 2 | | | | |
| Middle Occipital Gyrus | 5 | 1 | | | X | |
| Superior Parietal Lobule | 5 | 4 | | | X | |
| Angular Gyrus | 4 | 0 | | X | | |
| Inferior Frontal Gyrus (p. | 4 | 3 | | | | |
| Opercularis) | | | | | | |
| Mid Orbital Gyrus | 4 | 1 | | | | |
| Anterior Cingulate | 3 | 2 | | | | |
| Cortex | | | | | | |
| Caudate Nucleus | 3 | 0 | | | | |
| Middle Temporal Gyrus | 3 | 5 | | | | |
| Rectal Gyrus | 3 | 4 | | | | |
| SMA | 3 | 2 | | | X | |
| ParaHippocampal Gyrus | 2 | 2 | | | | |
| Rolandic Operculum | 2 | 4 | | X | | |
| Superior Frontal Gyrus | 2 | 3 | | | | |
| Temporal Pole | 2 | 0 | | | | |
| Hippocampus | 1 | 1 | | | | |
| Inferior Occipital Gyrus | 1 | 2 | | | | X |
| Insula Lobe | 1 | 0 | X | | | |
| Middle Cingulate Cortex | 1 | 4 | | | | |
| Middle Orbital Gyrus | 1 | 0 | X | | | |
| Putamen | 0 | 0 | | | X | |
| Superior Orbital Gyrus | 1 | 1 | | | | |
| Superior Temporal | 1 | 1 | X | | | |
| Gyrus | | | | | | |
| SupraMarginal Gyrus | 1 | 4 | | | | |
| Medial Temporal Pole | 0 | 3 | | | | |
| Olfactory cortex | 0 | 1 | | | | |
| Superior Occipital Gyrus | | | | X | | |
| Thalamus | | | | | X | |

‘L’ and ‘R’ indicate left and right hemispheres, respectively. ‘X’ indicates whether the region was also identified as effector-dependent or effector-independent by the voxelwise analysis.

### Discussion

This study used fMRI to assess whole-brain and cerebellar specific somatotopic brain activity while force production was precisely controlled by the foot, hand, and mouth. Other neuroimaging methods such as, e.g., EEG can also be utilized. Univariate and multivariate machine learning analyses were implemented to characterize, differentiate, and classify brain activation associated with each effector. Together the voxelwise and classification analyses identified effector-dependent activity in regions of the post-central gyrus, precentral gyrus, and paracentral lobule. Univariate voxelwise analyses identified middle occipital gyrus and putamen as regions showing effector-independent activation. Finally, SMA, regions of inferior and superior parietal lobule, and cerebellum each contained effector-dependent and effector-independent representations. Machine learning analyses showed that increasing the spatial resolution of the data-driven model increased classification accuracy, which reached 85-95% with supervoxel numbers of around 1000. The SLIC-based supervoxel parcellation outperformed classification analyses using established brain templates and random simulations. Occlusion experiments further demonstrated redundancy across the sensorimotor network when classifying effectors. The observations extend the understanding of effector-dependent and effector-independent organization within the human brain.

Within the cortex, the post-central gyrus, paracentral lobule, precentral gyrus and inferior and superior parietal lobe were most commonly identified as showing effector-dependent activation. Left post-central gyrus was the region most often identified across classification models and the center of mass of the largest cluster of activation in the voxelwise analysis was centered in the post-central gyrus. Clusters of activation in the post-central gyrus extended anteriorly into the precentral gyrus, while also conforming to the medial to lateral hand to mouth organization in sensorimotor cortex. The classification algorithm also exploited the BOLD signal in the medial wall of the pre- and post-central gyrus, with the paracentral lobule region being identified most often after the post-central gyrus. The paracentral lobule underlies sensorimotor function of the lower limbs, and was the second-largest cluster of activation identified in the univariate voxelwise analysis.

In humans, somatotopic organization is more discrete and segregated in S1 as compared to the more integrated and overlapping organization in M1, and the findings are consistent with this. One explanation for the repeated identification of the post-central gyrus across the analyses is sensory feedback. Using a population receptive field approach to differentiate within-limb somatotopy of finger digits, it has been demonstrated that receptive fields in M1 are larger compared to S1, such that sensory information occurs at a more specific level in S1 as compared to motor activity in M1. The demonstration of larger receptive fields with less segregation in precentral gyrus is consistent with primate studies showing extensive intrinsic horizontal connections in M1. Additional support for the findings comes from evidence showing that upper extremity nerve fibers are 75-95% sensory, with the ratio of sensory to motor fibers increasing when moving distally. As such, greater amounts of sensory feedback are needed to perform more precise movements, as was the case in the current study given the real-time visual feedback presented to the subjects. Previous somatotopy studies often use visual stimuli to cue individuals when to move which effector, but do not always provide real-time visual feedback to the subject as in the current study. Here, controlled and normalized target force amplitude across effectors can be provided using precise visual feedback and the literature extended by identifying a preference for between limb somatotopic organization in post-central gyrus and paracentral lobule as compared to precentral gyrus.

Both effector independent and effector dependent activation was identified in regions of SMA, IPL, and SPL. Effector-independent activation in SMA and bilateral middle-to-anterior intraparietal sulcus in the parietal lobe has been shown in individuals with congenitally absent upper limbs when pointing with their foot. IPL and SPL regions in the current study were within 5 mm of each other in the Y and Z direction and in close proximity to the intraparietal sulcus. In contrast, effector-dependent activation in IPL was more anterior and inferior and bordered the post-central gyrus. A similar pattern emerged in SPL with effector-dependent activity bordering the post-central gyrus in more anterior and superior regions compared to effector-independent activity. Somatotopic organization has also been shown in SMA, with a rostral-caudal face-to-foot organization, and in the putamen, with a ventral-to-rostral foot-to-face organization. Five of the classification models identified SMA as effector-dependent; however, these supervoxels were large (>450 voxels), and extended posteriorly into the paracentral lobule. Likewise, a single classification model identified the putamen as an effector-dependent region. The limited number of hits across classification models and the absence of voxelwise effector-dependent activation in these regions may be the result of large clusters and widespread activation across the visuomotor system associated with the implemented visuomotor task. Indeed, common activation was also evident in the middle occipital gyrus, consistent with other visuomotor studies that use real-time feedback. The paradigm differed from many other somatotopy studies in the extent to which the visuomotor network was engaged, and this may have masked the more fine-grained somatotopic organization that others have observed in these regions.

The observations reveal that the cerebellum contains both effector-dependent and effector-independent representations. The cerebellum was the third most commonly identified effector-dependent region. The majority of these regions were in the left hemisphere contralateral to the hand and foot, likely driven by the machine learning algorithm exploiting the bilateral activation associated with the mouth task (FIG. 5B). However, the voxelwise analysis did identify foot- and hand-related effector-dependent activity in right (ipsilateral) lobules IV-V, in addition to left lobule VI activity associated with the mouth task. While a similar anterior to posterior, foot-to-head organization has previously been shown for lobules II-VI, some evidence of effector-independent regions also exists. Effector-independent activity was found in left lobule VI (−25, −65, −26), in close proximity to the region of lobule VI that was previously identified as responding to multimodal pain and motor stimuli. Other evidence points to regions of Lobule VI being involved in early learning during explicit sequence learning tasks, suggesting more generalized effector and modality independent sensorimotor processing within this cerebellar region. Invasive and non-invasive stimulation studies in rodents and humans have targeted the cerebellum, and the findings add to the growing literature informing the stimulation of effector-dependent and effector-independent cerebellar regions.

The effector classification performance increased with an increasing number of supervoxels, reaching 94% accuracy with 10,000 specified supervoxels. The total number of supervoxels allowed across models was varied to identify the extent to which coarse to fine parcellation alters classification accuracy. As expected, coarser parcellation using fewer and larger supervoxels led to lower accuracy whereas finer parcellation using more and smaller supervoxels led to higher accuracy. The disclosed approach outperformed standardized brain atlases in classifying effectors. It is interesting to note that while all of three standardized brain atlases perform worse than the supervoxel-derived atlas, the Brainnetome atlas achieved the highest accuracy among the three standardized atlases. The Brainnetome includes subdivisions in the precentral and post-central gyrus for the hand and face region, trunk, upper limb, as well as lower limb subdivisions in the paracentral lobule. The AAL does not subdivide the precentral gyrus, but does include the paracentral lobule.

Finally, the Harvard atlas includes the entire pre- and post-central gyrus without any subdivisions. It is therefore not surprising that classification accuracy improves with increasing granularity, especially within the post-central gyrus, which across the models, was the most frequently identified region that contributed to the classification accuracy. The data demonstrate that predefined atlases lead to classification accuracies in the 70-80% range, whereas the current supervoxel approach reached 94%. A supervoxel approach has previously been used to segment and recognize 3D boundaries and shapes in mitochondria using electron microscopy, livers using volumetric CT, brain tumors in MRI, and resting state fMRI networks with good success. Here, for the first time, a supervoxel approach was implemented to classify movement of different effectors using fMRI data.

Both multivariate pattern analysis (MVPA) and classification approaches have been used to determine how well brain activity can distinguish different limbs performing similar movements, different movements performed by the same limb, and imagining as compared to executing movements. The frontoparietal motor network has been consistently identified across these studies as having the ability to distinguish between different movements. This is evident despite variations in the movements being made, the neuroimaging technique being employed, and the analytical approach being taken. Indeed, the redundancy in the system, in terms of how many areas show somatotopic organization, was confirmed in the occlusion analysis which demonstrated that even when removing the most commonly identified brain region, classification accuracy remained ˜80% or higher.

Many studies have used EEG measurements to classify movement, given its logistical and practical advantages in real-world settings as compared to the confines of fMRI. Cortical dynamics have been assayed using 64 channel EEG while subjects imagined or executed unimanual or bimanual hand and foot flexion. Classification at the individual level reached 69% which dropped to 56% at the group level. Individual-level classification accuracy reached 84% when differentiating left- and right-hand movements performed by one individual using EEG data collected from 2 electrodes. As one would expect, classification accuracy drops when the movements being made are subtler. Previous work required subjects to execute or imagine a right-hand squeeze, left-hand squeeze, press of the tongue on the roof of the mouth, or a right foot toe curl while EEG data were collected. Classification accuracy was at or below 60% for executed movements and at or below 50% for imagined movements. Inclusion of the tongue press may have attenuated classification accuracy, with tongue/lip/mouth movements being harder to classify as compared to movements of the hand and foot. Within-limb studies have also used EEG to show that forward reaching movements to different targets can be classified with between 50-80% accuracy depending on the number of targets being decoded and the number of subjects being tested. In each case, signals over parietal cortex prominently contributed to classification accuracy, consistent with the observations. Frontal and parietal brain areas have also been identified in fMRI studies that have classified eye and hand movements with ˜55-65 accuracy, and the planning and execution of reaching and grasping movements with the left and right hand with 55-70% accuracy. Executing, observing, and imagining reaching movements of the same limb has been classified with 80% and within-subject mouth movements with 90% accuracy. Other fMRI studies have implemented MVPA approaches within specific regions such as M1 during hand and foot rotation. General linear modeling approaches have also been implemented to determine differences in brain activation during flexion of the right finger, elbow, and ankle. The current literature was extended by using fMRI data and a generalizable classifier to differentiate three different effectors performing the same task with the same relative amount of force. The novel approach achieved 85-95% accuracy. This is notable because a whole-brain analysis approach has been implemented that also allowed us to vary the number of supervoxels across different models, while also using a between—rather than within-subjects classification approach to increase the generalizability of the findings.

In addition to the well-established somatotopy in the pre- and post-central gyrus, there is now strong evidence that somatotopic organization is evident across other regions in the sensorimotor network. This raises several experimental questions: To what extent is activation in the sensorimotor network effector-dependent and effector-independent? How important is the sensorimotor cortex when predicting the motor effector? Is there redundancy in the distributed somatotopically organized network such that removing one region has little impact on classification accuracy? To answer these questions, a novel approach was developed. fMRI data were collected while human subjects performed a precisely controlled force generation task separately with their hand, foot, and mouth. A simple linear iterative clustering (SLIC) algorithm was used to segment whole-brain beta coefficient maps to build an adaptive brain parcellation and then classified effectors using extreme gradient boosting (XGBoost) based on parcellations at various spatial resolutions. This allowed us to understand how data-driven adaptive brain parcellation granularity altered classification accuracy. Results revealed effector-dependent activity in regions of the post-central gyrus, precentral gyrus, and paracentral lobule. SMA, regions of inferior and superior parietal lobule, and cerebellum each contained effector-dependent and effector-independent representations. Machine learning analyses showed that increasing the spatial resolution of the data-driven model increased classification accuracy, which reached 85-95% with supervoxel numbers of around 1000. The SLIC-based supervoxel parcellation outperformed classification analyses using established brain templates and random simulations. Occlusion experiments further demonstrated redundancy across the sensorimotor network when classifying effectors.

It should be emphasized that the above-described embodiments of the present disclosure are merely possible examples of implementations set forth for a clear understanding of the principles of the disclosure. Many variations and modifications may be made to the above-described embodiment(s) without departing substantially from the spirit and principles of the disclosure. All such modifications and variations are intended to be included herein within the scope of this disclosure and protected by the following claims.

The term “substantially” is meant to permit deviations from the descriptive term that don't negatively impact the intended purpose. Descriptive terms are implicitly understood to be modified by the word substantially, even if the term is not explicitly modified by the word substantially.

It should be noted that ratios, concentrations, amounts, and other numerical data may be expressed herein in a range format. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a concentration range of “about 0.1% to about 5%” should be interpreted to include not only the explicitly recited concentration of about 0.1 wt % to about 5 wt %, but also include individual concentrations (e.g., 1%, 2%, 3%, and 4%) and the sub-ranges (e.g., 0.5%, 1.1%, 2.2%, 3.3%, and 4.4%) within the indicated range. The term “about” can include traditional rounding according to significant figures of numerical values. In addition, the phrase “about ‘x’ to ‘y’” includes “about ‘x’ to about ‘y’”.

1. A method for functional task prediction with dynamic supervoxel parcellation, comprising:
preprocessing activation data obtained from brains of multiple subjects to generate one or more dynamic parcellated supervoxel maps based on a whole brain map of the multiple subjects, the activation data associated with a functional task; and
determining an anatomical location of the functional task in a brain of another subject based upon classification of supervoxels of the one or more dynamic parcellated supervoxel maps.

2. The method of claim 1, wherein the preprocessing comprises:
registering and averaging the activation data of the multiple subjects to produce the whole brain map; and
generating the one or more dynamic parcellated supervoxel maps from the whole brain map.

3. The method of claim 2, wherein supervoxels of the one or more dynamic parcellated supervoxel maps are identified from the whole brain map.

4. The method of claim 3, wherein the supervoxels are identified using a 1-way ANOVA analysis.

5. The method of claim 2, comprising masking the one or more dynamic parcellated supervoxel maps using a conjunction map.

6. The method of claim 5, wherein the one or more dynamic parcellated supervoxel maps comprises average beta coefficients.

7. The method of claim 1, wherein the functional task is associated with a foot, a hand, or a mouth of the subject.

8. The method of claim 1, wherein the activation data is acquired through magnetic resonance imaging of the subject.

9. The method of claim 1, wherein the classification of the supervoxels comprises generating weights for the supervoxels using machine learning.

10. The method of claim 9, wherein the machine learning comprises gradient boosting decision trees, artificial neural networks, or support vector machines.

11. The method of claim 9, wherein the anatomical location of the functional task is determined based upon the generated weights.

12. A system, comprising:
at least one computing device comprising processing circuitry including a processor and memory, the at least one computing device configured to at least:
preprocess activation data obtained from brains of multiple subjects to generate one or more dynamic parcellated supervoxel maps based on a whole brain map of the multiple subjects, the activation data associated with a functional task; and
determine an anatomical location of the functional task in a brain of another subject based upon classification of supervoxels of the one or more dynamic parcellated supervoxel maps.

13. The system of claim 12, wherein preprocessing the activation data comprises:
registering and averaging the activation data of the multiple subjects to produce the whole brain map; and
generating the one or more dynamic parcellated supervoxel maps from the whole brain map.

14. The system of claim 13, wherein preprocessing further comprises masking the one or more dynamic parcellated supervoxel maps using a conjunction map.

15. The system of claim 14, wherein the one or more dynamic parcellated supervoxel maps comprises average beta coefficients.

16. The system of claim 12, wherein the activation data is acquired through magnetic resonance imaging of the subject while carrying out the functional task.

17. The system of claim 16, wherein the functional task is associated with a foot, a hand, or a mouth of the subject.

18. The system of claim 12, wherein the classification of the supervoxels comprises generating weights for the supervoxels using machine learning.

19. The system of claim 18, wherein the machine learning comprises gradient boosting decision trees, artificial neural networks, or support vector machines.

20. The system of claim 19, wherein the anatomical location of the functional task is determined based upon the generated weights.
